(19) United States
(12) Patent Application Publication
(10) Pub. No.: US 2024/0390529 A1
(43) Pub. Date: Nov. 28, 2024

(54) DUAL-MODE PROBE FOR DETECTING HYDROGEN SULFIDE AND USE THEREOF

(71) Applicant: KYUNGPOOK NATIONAL UNIVERSITY INDUSTRY-ACADEMIC COOPERATION FOUNDATION, Daegu (KR)

(72) Inventors: Jeongsoo YOO, Daegu (KR); Jeong Eun LIM, Daegu (KR)

(73) Assignee: KYUNGPOOK NATIONAL UNIVERSITY INDUSTRY-ACADEMIC COOPERATION FOUNDATION, Daegu (KR)

(21) Appl. No.: 18/776,162

(22) Filed: Jul. 17, 2024

Related U.S. Application Data
(63) Continuation-in-part of application No. PCT/KR2023/000824, filed on Jan. 17, 2023.

(30) Foreign Application Priority Data
Jan. 17, 2022 (KR) 10-2022-0006610

(51) Int. Cl. A61K 51/04; A61B 6/00; C07B 59/00; C07F 1/08

(52) U.S. Cl. CPC A61K 51/0478; A61B 6/481; C07B 59/002; C07F 1/08; A61K 2123/00; C07B 2200/05

(57) ABSTRACT

The present invention relates to a dual-mode probe for detecting of hydrogen sulfide and use thereof and, more specifically to a dual-mode probe that has excellent blood-brain barrier permeability and is capable of fluorescence and nuclear imaging and a use thereof for detecting hydrogen sulfide.

### TECHNICAL FIELD

The present application claims priority to Korean Patent Application No. 2022-0006610 filed on Jan. 17, 2022, and the entire specification thereof is incorporated herein by reference.

The present invention relates to a dual-modality probe for detection of hydrogen sulfide and use thereof and, more specifically to a dual-modality probe which has excellent blood-brain barrier permeability and is capable of fluorescence and nuclear imaging and use thereof in detecting hydrogen sulfide.

### BACKGROUND ART

According to recent research results, hydrogen sulfide (H₂S), along with nitric oxide and carbon monoxide, is known to participate in various physiological phenomena as a 3rd gasotransmitter and gasomediator.

The concentration of H₂S in blood plasma is known to be around 50 to 100 μM. Recent studies have reported that H₂S acts as an important signaling substance in various physiological actions such as various inflammatory reactions, cardiovascular diseases, vasodilation, glucose metabolism, and new blood vessel formation.

Accordingly, various technologies that can detect and quantify hydrogen sulfide are being developed competitively, and in particular, the importance of developing a method to detect hydrogen sulfide in the body using non-invasive imaging is emerging.

Focusing on the role of hydrogen sulfide in diagnosis of diseases and participation in physiological phenomena, various types of markers are currently being introduced. However, it is known to be very difficult and complicated to detect and accurately quantify hydrogen sulfide because it requires some conditions, e.g., being capable of measuring H₂S in the liquid state, having selectivity between other anionic species, and having selectivity with reduced glutathione (GSH).

In this regard, up to now, a detection method using a chromophore that utilizes the chemical properties of hydrogen sulfide in cytosol within cells and blood plasma, a detection method passing through an electrode specific for sulfide ions, and a detection method using gas chromatography have mostly been used, and various fluorescent probe methods have recently been developed. However, due to the limitations of the fluorescent probe methods, detection of hydrogen sulfide through imaging is very limited in small animals. Thus, actual biological imaging and research on life phenomena are bound to be extremely limited.

Meanwhile, in the brain, H₂S plays an important role in memory and cognition. In addition, its antioxidant, anti-inflammatory, anti-apoptotic and additional effects are associated with neurological disorders. Disruption of endogenous H₂S levels is associated with neurodegenerative disorders such as Alzheimer's disease and Parkinson's disease. Recent studies have shown that H₂S has therapeutic potential as a neuroprotectant in neurological diseases.

It is important to accurately measure H₂S concentrations in the brain to study the mechanisms underlying the various pathophysiological effects of H₂S and to identify new roles for H₂S in the brain of a subject. Typically, several methods such as spectroscopy, chromatography and electrochemical methods are used to quantify H₂S, but they do not correctly reflect the H₂S level in biological samples. All these methods are invasive and cannot monitor H₂S concentrations non-invasively in living cells and animals. Over the past decade, there have been notable advances in H₂S biosensing using fluorescent probes. Highly selective and sensitive fluorescence imaging probes have been developed by taking advantage of the diverse chemical properties of H₂S. Submicromolar H₂S concentrations inside living organelles were sensitively detected at high resolution by fluorescence microscopy. However, due to the unique characteristic of optical imaging, i.e., short penetration into tissue (typically less than a few millimeters), even the internal organs of small mice cannot be accurately imaged by using fluorescent probes. The brain, safely located inside the rigid skull, is one of the most challenging organs for optical imaging.

### SUMMARY OF INVENTION

### Technical Problem

In the present invention, the present inventor continued research to develop an imaging probe capable of detecting hydrogen sulfide in each organ of the body, especially the brain, and, as a result, discovered that a compound in which a radioisotope copper ligand and a fluorescent substance are combined and a complex compound containing copper exhibit surprisingly excellent brain distribution and can be used as a dual-modality contrast agent capable of simultaneous fluorescence and nuclear imaging, and thus the present invention has been completed.

Therefore, the object of the present invention is to provide a complex compound into which the radioisotope Cu is introduced, defined by the following formula 1:

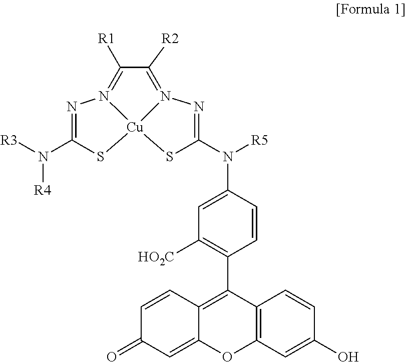

wherein R1 to R5 are each independently hydrogen or C1-C10 straight or branched alkyl, and Cu is ⁶⁰Cu, ⁶¹Cu, ⁶²Cu, ⁶⁴Cu, or ⁶⁷Cu; or a pharmaceutically acceptable salt thereof.

Another object of the present invention is to provide a contrast agent comprising the complex compound or pharmaceutically acceptable salt thereof.

Another object of the present invention is to provide a composition for diagnosing inflammatory diseases, comprising the complex compound or pharmaceutically acceptable salt thereof.

Another object of the present invention is to provide a composition for diagnosing inflammatory diseases, consisting of the complex compound or pharmaceutically acceptable salt thereof.

Another object of the present invention is to provide a composition for diagnosing inflammatory diseases, essentially consisting of the complex compound or pharmaceutically acceptable salt thereof.

Another object of the present invention is to provide use of the complex compound or pharmaceutically acceptable salt thereof in preparing a composition for diagnosing inflammatory diseases.

Another object of the present invention is to provide a method for diagnosing inflammatory diseases, comprising (a) administering the complex compound or pharmaceutically acceptable salt thereof to a subject; (b) scanning the subject by using a fluorescence imaging device, a radioactive imaging device, or a combination thereof; and (c) analyzing the scanned image and diagnosing the subject as having an inflammatory disease when a fluorescence signal, a radiation signal, or a combination thereof is increased, as compared to the control group.

### Solution to Problem

In order to achieve the above-described object of the present invention, the present invention provides a complex compound into which the radioisotope Cu is introduced, defined by the following formula 1:

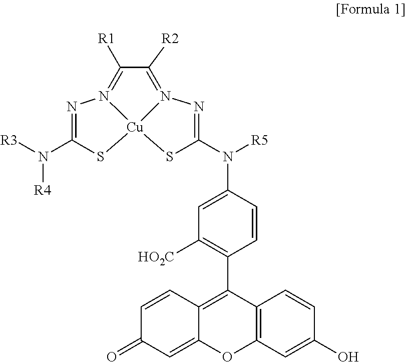

wherein R1 to R5 are each independently hydrogen or C1-C10 straight or branched alkyl, and Cu is ⁶⁰Cu, ⁶¹Cu, ⁶²Cu, ⁶⁴Cu, or ⁶⁷Cu; or a pharmaceutically acceptable salt thereof.

In order to achieve another object of the present invention, the present invention provides a contrast agent comprising the complex compound or pharmaceutically acceptable salt thereof.

In order to achieve another object of the present invention, the present invention provides a composition for diagnosing inflammatory diseases, comprising the complex compound or pharmaceutically acceptable salt thereof.

In addition, the present invention provides a composition for diagnosing inflammatory diseases, consisting of the complex compound or pharmaceutically acceptable salt thereof.

In addition, the present invention provides a composition for diagnosing inflammatory diseases, essentially consisting of the complex compound or pharmaceutically acceptable salt thereof.

In order to achieve another object of the present invention, the present invention provides use of the complex compound or pharmaceutically acceptable salt thereof in preparing a composition for diagnosing inflammatory diseases.

In order to achieve another object of the present invention, the present invention provides a method for diagnosing inflammatory diseases, comprising (a) administering the complex compound or pharmaceutically acceptable salt thereof to a subject; (b) scanning the subject by using a fluorescence imaging device, a radioactive imaging device, or a combination thereof; and (c) analyzing the scanned image and diagnosing the subject as having an inflammatory disease when a fluorescence signal, a radiation signal, or a combination thereof is increased, as compared to the control group.

Hereinafter, the present invention will be described in detail.

The present invention provides a complex compound into which the radioisotope Cu is introduced, defined by the following formula 1:

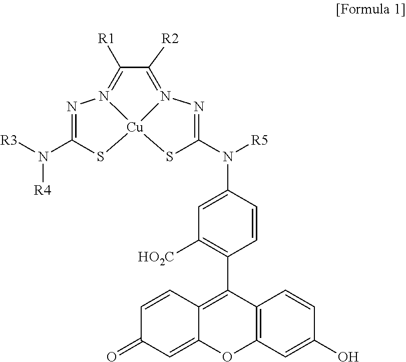

wherein R1 to R5 are each independently hydrogen or C1-C10 straight or branched alkyl, and Cu is ⁶⁰Cu, ⁶¹Cu, ⁶²Cu, ⁶⁴Cu, or ⁶⁷Cu; or a pharmaceutically acceptable salt thereof.

According to one embodiment of the present invention, R1 to R5 in formula 1 may each independently be hydrogen or C1-C5 straight or branched alkyl.

According to one embodiment of the present invention, R1 to R3 in formula 1 may each independently be C1-C5 straight or branched alkyl, and R4 and R5 may be hydrogen.

According to one embodiment of the present invention, R1 to R3 in formula 1 may be C1-C5 straight or branched chain alkyl, and R4 and R5 may be hydrogen.

According to one embodiment of the present invention, R1 to R3 in formula 1 may be methyl, and R4 and R5 may be hydrogen.

In the present invention, Cu may be ⁶⁰Cu, ⁶¹Cu, ⁶²Cu, ⁶⁴Cu, or ⁶⁷Cu, preferably ⁶⁴Cu, but the present invention is not limited thereto.

In the present invention, the term “a complex compound” may be understood to have the same meaning as a complex, and may refer to a structure consisting of a central atom or ion, and a molecule or anion (specifically, a ligand) surrounding same while coordinating with same.

The radioactive isotope Cu of the complex compound provided by the present invention is separated from the complex compound in vivo, selectively forming hydrogen sulfide and copper sulfide (CuS), and gamma rays are emitted from the copper sulfide to enable nuclear imaging. At the same time, the fluorescent structure of the complex provided by the present invention enables fluorescence imaging.

According to one embodiment of the present invention, real-time observation was performed in animal models by using fluorescence and nuclear medicine imaging using the complex compound of formula 1, and, as a result, it was confirmed that the complex compound according to the present invention selectively bound to hydrogen sulfide and had a fast reaction rate with hydrogen sulfide. As such, hydrogen sulfide can selectively image areas where is abnormally increased within cells or tissues and does not affect the anatomical characteristics of the body part, such that diseases in completely unexpected areas can be detected, and the problem that tests can be performed after a certain period of time has passed after conventional contrast agents are administered can be solved. In particular, the complex compound of formula 1 according to the present invention exhibits appropriate lipophilicity to penetrate the blood-brain barrier to have a particularly high distribution in the brain among various organs of the body, and has a high residence time to be useful for detecting hydrogen sulfide in the brain of a subject.

In one aspect of the present invention, the lipophilicity log D7.4 of the complex according to formula 1 may be 1.5 to 3.0, preferably 1.5 to 2.5, and more preferably 1.5 to 2.0, and, therefore, the blood-brain barrier permeability may be very excellent.

In addition, the complex compound into which the radioactive isotope Cu according to formula 1 is introduced can be used as a probe for detecting hydrogen sulfide to image sulfide ions in cells and extracellular matrix localized to locations or tissues where hydrogen sulfide is abnormally increased after its administration into the body.

As used herein, the term “pharmaceutically acceptable” refers to a compound or composition that has a reasonable benefit/risk ratio without undue toxicity, irritation, allergic reactions or other problems or complications and, thus, is suitable for use in contact with the tissues of a subject (e.g., a human being), and is within the scope of sound medical judgment.

The complex compound defined by formula 1 of the present invention may be used in the form of a pharmaceutically acceptable salt, and an acid addition salt formed by a pharmaceutically acceptable free acid may be useful as the salt.

Acid addition salts may be obtained from inorganic acids such as hydrochloric acid, nitric acid, phosphoric acid, sulfuric acid, hydrobromic acid, hydroiodic acid, nitrous acid, and phosphorous acid, and non-toxic organic acids such as aliphatic mono and dicarboxylates, phenyl-substituted alkanoates, hydroxy alkanoates and alkanedioates, aromatic acids, and aliphatic and aromatic sulfonic acids.

These pharmaceutically non-toxic salts comprise sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methyl benzoate, dinitro benzoate, hydroxybenzoate, methoxybenzoate, phthalate, terephthalate, Benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate or mandelate.

The acid addition salt according to the present invention may be prepared by conventional methods, for example, dissolving the compound of formula 1 in an aqueous acid solution, and precipitating the salt using water-miscible organic solvents such as methanol, ethanol, acetone and acetonitrile. In addition, it may also be prepared by evaporating the solvent or excess acid from the mixture, and drying same or suction-filtering the precipitated salt.

In addition, a pharmaceutically acceptable metal salt may be prepared using a base. Alkali metals or alkaline earth metal salts are obtained by for example, dissolving the compound of formula 1 in an excess of alkali metal hydroxide or alkaline earth metal hydroxide solution, filtering the insoluble compound salt, and evaporating and drying the filtrate. At this time, it may be pharmaceutically appropriate to prepare sodium, potassium or calcium salts as metal salts. The corresponding silver salts are obtained by reacting an alkali metal or alkaline earth metal salt with a suitable silver salt (e.g., silver nitrate).

The present invention also provides a contrast agent comprising the complex compound or pharmaceutically acceptable salt thereof.

The type of the contrast agent is not particularly limited, but may be characterized that the contrast agent is one or more selected from the group consisting of a contrast agent for positron emission tomography (PET), a contrast agent for fluorescence imaging, a contrast agent for gamma cameras, a contrast agent for single photon emission computed tomography (SPECT), a Cherenkov optical imaging contrast agent, and a contrast agent for charge-coupled device (CCD), and can be used as a disease diagnostic tool, such as an imaging composition and imaging method.

Preferably, the contrast agent may be a contrast agent for positron emission tomography (PET) and/or a contrast agent for fluorescence imaging, and may be characterized as being a dual-modality contrast agent capable of simultaneous PET and fluorescence imaging.

The contrast agent provided by the present invention can react specifically with hydrogen sulfide to enable imaging, as described above, such that it can be used as a contrast agent for detecting hydrogen sulfide in the body of a subject, and is preferably used as a contrast agent for detecting hydrogen sulfide in the brain due to its high brain penetration properties.

In actual use, the contrast agent of the present invention may be combined with a pharmaceutically acceptable carrier according to conventional pharmaceutical preparation techniques. The carrier may take a wide variety of forms depending on the preparation method preferred for, e.g., oral or parenteral administration (such as intravenous administration).

In addition, the contrast agent of the present invention may be administered at a dosage of 0.1 mg/kg to 1 g/kg, and more preferably at a dosage of 0.1 mg/kg to 500 mg/kg. Meanwhile, the dosage may be appropriately adjusted within the range of radiation exposure allowed daily or annually depending on the patient's age, gender, and condition.

The contrast agent of the present invention may further comprise inert ingredients such as a pharmaceutically acceptable carrier. As used herein, “pharmaceutically acceptable carrier” is a term referring to a composition, specifically, a component other than the active substance of a pharmaceutical composition. Examples of pharmaceutically acceptable carriers comprise binders, disintegrants, diluents, fillers, lubricants, solubilizers or emulsifiers, and salts.

The contrast agent may be administered to a subject by parenteral administration, and the parenteral administration may be intravenous injection, intraperitoneal injection, intramuscular injection, or subcutaneous injection, but intravenous administration may be most preferred.

The present invention also provides a composition for diagnosing inflammatory diseases comprising the complex compound or pharmaceutically acceptable salt thereof.

The inflammatory diseases are preferably those at which site the concentration of hydrogen sulfide is increased, and non-limiting examples thereof may comprise neuroinflammatory diseases, rheumatoid arthritis, non-rheumatic inflammatory arthritis, arthritis related to Lyme disease, inflammatory osteoarthritis, meningitis, osteomyelitis, inflammatory bowel disease, appendicitis, pancreatitis, sepsis, pyelitis, nephritis, and inflammation diseases due to bacterial infection.

In the present invention, the types of neuroinflammatory diseases are not particularly limited, but may comprise Alzheimer's disease, vascular dementia, frontotemporal dementia, alcoholic dementia, Parkinson's disease, traumatic brain injury, Niemann-Pick disease, amyotrophic axial sclerosis, multiple sclerosis, Huntington's disease, Creutzfeldt-Jakob disease and stroke. Regarding neuroinflammatory diseases known to be related to hydrogen sulfide, information known in the art may be referred to.

In the present invention, the types of heart diseases are not particularly limited, but may comprise myocardial infarction, cardiac ischemia, angina, cardiomyopathy, and endocarditis.

The present invention also provides a method for diagnosing inflammatory diseases in the body, comprising providing the complex compound of formula 1 into which the radioisotope Cu is introduced within a pharmacological carrier; injecting the complex into a mammal; and scanning the mammal by using a radioactive diagnostic imaging device and a fluorescent imaging device.

The mammal may be a human being or mammals other than human beings.

The present invention provides use of the complex compound or pharmaceutically acceptable salt thereof in preparing a composition for diagnosing inflammatory diseases.

The present invention provides method for diagnosing inflammatory diseases, comprising (a) administering the complex compound or pharmaceutically acceptable salt thereof to a subject; (b) scanning the subject by using a fluorescence imaging device, a radioactive imaging device, or a combination thereof; and (c) analyzing the scanned image and diagnosing the subject as having an inflammatory disease when a fluorescence signal, a radiation signal, or a combination thereof is increased, as compared to the control group.

In one embodiment, the invention provides a method of diagnosing and treating an inflammatory disease in an individual comprising (i) administering the complex compound or pharmaceutically acceptable salt thereof to a subject; (ii) scanning the subject by using a fluorescence imaging device, a radioactive imaging device, or a combination thereof; (iii) analyzing the scanned image and diagnosing the subject as having an inflammatory disease when a fluorescence signal, a radiation signal, or a combination thereof is increased, as compared to the control group; and (iv) administering a therapeutic drug for treating an inflammatory disease to the diagnosed subject or treating the disease through surgery.

The method comprising steps (i) to (iv) are understood based on the method comprising steps (a) to (c) described above.

The step (iv) comprises administering therapeutic drugs such as aspirin, betamethasone, vedolizumab and Natalizumab to the subject diagnosed with the disease in step (iii) and treating the disease through means such as surgery.

The ‘treatment’ of the present invention refers generically to improving an inflammatory disease or the symptoms of the disease, which may comprise curing or substantially preventing the disease, or improving the condition thereof, and comprises alleviating, curing, or preventing one or most symptoms resulting from the disease, but the present invention is not limited thereto.

The ‘sample’ of the present invention is obtained separately from an individual suspected of having a disease, and may be selected from the group consisting of cells, tissues, blood, serum, plasma, saliva, and sputum, mucosal fluid and urine, but the present invention is not limited thereto. In addition, the ‘individual’ or ‘subject’ may be an animal, preferably a mammal, especially an animal comprising a human being, and may also be a cell, a tissue or an organ derived from an animal. The subject may be a patient in need of the treatment effect.

As used herein, the term “comprising” is used to have the same meaning as “including” or “characterized by.” In the composition or method according to the present invention, additional components or steps of the method not specifically mentioned are not excluded. In addition, the term “consisting of” indicates that additional elements, steps, or ingredients not separately described are excluded. The term “essentially consisting of” indicates that, in addition to the substances or steps described, substances or steps that do not substantially affect the basic properties thereof may fall within the scope of the composition or method.

### Advantageous Effects of Invention

The complex compound provided by the present invention can selectively bind to hydrogen sulfide to selectively image areas where hydrogen sulfide is abnormally increased within cells or tissues. In particular, it has a high blood-brain barrier permeability to very effectively detect brain hydrogen sulfide, which is detected at high levels. In addition, the complex compound provided by the present invention can be used as a dual-modality contrast agent capable of simultaneous nuclear and fluorescence imaging and, therefore, can be used for diagnosis and research purposes of various diseases mediated by hydrogen sulfide.

### BRIEF DESCRIPTION OF DRAWINGS

FIG. 1 is a diagram showing the synthesis process of Cu-ASTM-FITC according to the present invention.

FIG. 2 shows ¹H NMR spectrum data of ATSM-FITC(4).

FIG. 3 shows ¹³C NMR spectrum data of ATSM-FITC(4).

FIG. 4 shows HRMS spectrum data of ATSM-FITC(4).

FIG. 5 shows HRMS spectrum data of Cu-ATSM-FITC(5).

FIGS. 6A to 6D show experimental results on the sensitivity and selectivity of Cu-ATSM-FITC(5).

FIG. 6A Absorbance and emission spectra of Cu-ATSM-FITC(5) in PBS (10% DMSO, pH 7.4).

FIG. 6B Fluorescence spectrum of probe Cu-ATSM-FITC(5) (10 μM) after reaction with various concentrations of H₂S (0-100 μM).

FIG. 6C Results of confirming the fluorescence intensity over time of Cu-ATSM-FITC(5) in the presence of NaHS, L-Cys, and GSH.

FIG. 6D Selectivity of Cu-ATSM-FITC(5) toward various biological competitors comprising H₂S.

FIG. 7 is a diagram showing the mechanism by which Cu-ATSM-FITC(5) reacts with H₂S to exhibit fluorescence activity.

FIG. 8 shows experimental results of confirming the detection limit of Cu-ATSM-FITC (5) in the presence of H₂S.

FIG. 9 shows the results of calculating the quantum yield of ATSM-FITC(4) by comparison with the fluorescein standard value according to the equation.

FIG. 10 shows the results of confirming the change in fluorescence intensity of Cu-ATSM-FITC(5) in the presence or absence of H₂S at various pH values.

FIGS. 11A to 11C show the results of in vitro evaluation of the toxicity of Cu(ATSM-FITC)(5) complex ((FIG. 11A) human cervical cancer HeLa cells; (FIG. 11B) human liver cancer cell line (HepG2); and (FIG. 11C) human embryonic kidney 293 (HEK293) cells).

FIGS. 12A to 12D show the results of in vitro MTT assay evaluation of the toxicity of CuS to various cell lines ((FIG. 12A) HeLa; (FIG. 12B) human embryonic kidney 293 (HEK293); (FIG. 12C) human liver cancer cells (HepG2), and (FIG. 12D) human brain glioblastoma cells (U87MG)).

FIG. 13 shows the results of histological H&E staining of the mouse brain cerebral cortex administered with a high dose of Cu(ATSM-FITC)(5).

FIG. 14 shows the results of treating live HeLa cells with Cu-ATSM-FITC(5) and detecting exogenous and endogenous H₂S through fluorescence imaging.

FIGS. 15A to 15C show a schematic diagram showing the process in which ⁶⁴Cu-ATSM-FITC(6) radiolabeled with Cu-64 reacts with H₂S to decomplex, and ⁶⁴CuS is precipitated (FIG. 15A); the UV-HPLC profile (black) of Cu-ATSM-FITC(5) and the radio-HPLC profile (red) of ⁶⁴Cu-ATSM-FITC(6) (FIG. 15B); and the decomplexation profile of ⁶⁴Cu-ATSM-FITC(6) reacted with various concentrations of NaHS (FIG. 15C).

FIG. 16 shows the results of evaluation of the stability of [64Cu][Cu(ATSM-FITC)](6) in PBS and FBS at 37° C.

FIGS. 17A to 17H are diagrams showing the results of in vivo detection of endogenous H₂S in the mouse brain by ⁶⁴Cu-ATSM-FITC(6).

FIG. 17A Results of confirming distribution by tissue at the indicated time points after administration of ⁶⁴Cu-ATSM-FITC(6) in normal BALB/c mice.

FIGS. 17B to 17E PET/CT images 1 hour after administration of ⁶⁴Cu-ATSM-FITC(6).

FIG. 17F PET/CT images 1 hour after administering ⁶⁴Cu-ATSM-FITC(6) to mice treated with AOAA, an H₂S inhibitor.

FIG. 17G Results of quantifying the amount absorbed 1 hour after administration of ⁶⁴Cu-ATSM-FITC(6) in the brains of normal mice and AOAA-treated mice.

FIG. 17H A diagram showing the results of quantifying the concentration of H₂S in the brains of normal mice and AOAA-treated mice by using the methylene blue method and the results of relatively quantifying same based on biodistribution according to the present invention.

FIGS. 18A to 18C are diagrams showing the PET/CT images of the coronal section (FIG. 18A), sagittal section (FIG. 18B), and transverse (FIG. 18C) section of the brain 1 hour after administration of ⁶⁴Cu-ATSM-FITC(6) to SD-rats.

FIGS. 19A to 19D are diagrams showing the results of detecting H₂S by nuclear medicine imaging in a neuroinflammation animal model using ⁶⁴Cu-ATSM-FITC (6).

FIG. 19A A schematic diagram showing a method of inducing a neuroinflammatory animal model by administering LPS to the right hemisphere of the mouse brain.

FIG. 19B Results of brain PET imaging 10 minutes after administration of ⁶⁴Cu-ATSM-FITC(6) to control and LPS-treated animal models (white arrow: LPS administration site).

FIG. 19C Results of quantifying ⁶⁴Cu-ATSM-FITC(6) in the brains of the control and LPS-treated animal models.

FIG. 19D Results of quantifying H₂S in the brains of control and LPS-treated animal models according to the methylene blue method immediately after PET imaging.

FIG. 20 is a schematic diagram showing the process of synthesizing ATSM-aniline (9) and radiolabeling same with Cu-64 to produce ⁶⁴Cu-ATSM-aniline(10).

FIG. 21 shows the results of confirming the biodistribution 4 hours after administration of ⁶⁴Cu-ATSM-aniline(10) to normal BALB/c mice.

FIG. 22 shows radio-TLC profiles of ⁶⁴Cu-ATSM-FITC(6), ⁶⁴CuS, and a reaction mixture of ⁶⁴Cu-ATSM-FITC and H₂S that the decomplexation percentage was calculated using the peak integration ratios of ⁶⁴Cu-ATSM-FITC and ⁶⁴CuS. Radio-TLC conditions: silica, MeOH:EtOAc=5:95.

FIG. 23 shows coronal PET/CT images acquired 1 hour after administration of ⁶⁴Cu-ATSM-FITC(6) to a control (Normal rat) and a Parkinson's disease rat model.

### MODE FOR INVENTION

Hereinafter, the present invention will be described in detail by the following embodiments. However, the following embodiments are only for illustrating the present invention, and the present invention is not limited thereto.

### Experimental Method

### 1. Synthesis of 2-(6-hydroxy-3-oxo-3H-xanthen-9-yl)-5-((E)-2-((E)-3-(2-(methylcarbamothioyl)hydrazono)butan-2-ylidene)hydrazinecarbothioamido)benzoic Acid (ATSM-FITC, 4)

Compound 3 (191 mg, 1.02 mmol) in methanol (15 mL) was added to a methanol solution of FITC-NCS (380 mg, 1.02 mmol, 15 mL), and the mixture was stirred in the dark room at room temperature. The reaction was monitored by TLC (silica, MeOH:CH₂Cl₂=5:95) and confirmed to be completed within 30 minutes. It was subjected to concentration under reduced pressure to obtain a light yellow solid and purified by column chromatography to obtain ATSM-FITC(4) as a yellow solid (303 mg, 52.7%).

¹H NMR (500 MHz; DMSO-d₆): δ 2.28 (s, 3H), 2.32 (s, 3H), 3.05 (d, 3H, J=4.5 Hz), 6.57-6.62 (m, 4H), 6.69 (s, 1H), 6.70 (s, 1H), 7.27 (d, 1H, J=8.5 Hz), 8.02 (dd, 1H, J=2 Hz, J=8.5 Hz), 8.35 (d, 1H, J=2 Hz), 8.44-8.43 (m, 1H), 10.14 (s, 2H), 10.23 (s, 1H), 10.34 (s, 1H), 10.87 (s, 1H) ppm; ¹³C NMR (125 MHz; DMSO-d₆): δ 12.26, 12.68, 31.71, 83.54, 102.76, 110.00, 113.10, 120.23, 124.12, 126.60, 129.42, 133.02, 141.10, 148.10, 149.39, 150.55, 152.30, 159.96, 168.87, 177.15, 178.99 ppm; HRMS (FAB) m/z calcd. 577.1332 for C₂₇H₂₄N₆O₅S₂ [M+H]⁺, found 577.1332.

### 2. Synthesis of Copper(II) Complex of 2-(6-hydroxy-3-oxo-3H-xanthen-9-yl)-5-((E)-2-((E)-3-(2-(methylcarbamothioyl)hydrazono)butan-2-ylidene)hydrazinecarbothioamido)benzoic Acid (Cu-ATSM-FITC (5))

CuCl₂·2H₂O (46 mg, 0.27 mmol) in methanol (1 mL) was added to a solution of ATSM-FITC(4) (103 mg, 0.18 mmol). A dark brown precipitate was formed immediately. The mixture was then heated at reflux for 2 hours. It was cooled to room temperature, and the precipitate was collected, washed with methanol (3×1 mL), and dried under vacuum to obtain Cu-ATSM-FITC(5) as a brown solid (38 mg, 33%). The purity of the compound was confirmed by an HPLC profile (Waters, Grace smart RP C18 column [4.6 mm×250 mm, 5 μm], isocratic mobile phase MeOH:H₂O=60:40, flow rate of 1 mL/min), and identified by HRMS. HRMS (FAB) m/z calcd. 638.0462 for C₂₇H₂₂CuN₆O₅S₂ [M+H]⁺, found 638.0470.

### 3. Radiolabeling of ATSM-FITC(4) Using ⁶⁴Cu

Stock solutions of chelators (1 μg/μL) were prepared in anhydrous DMSO. Complexation of ⁶⁴Cu with ATSM-FITC(4) was performed by mixing ⁶⁴CuCl₂ (18.5 to 111 MBq) to which a carrier was not added in 0.01 M HCl (1 to 5 μL) with a chelator solution (10 to 20 μg) in 0.1 M ammonium acetate (pH 6.8, 100 μL). It was subjected to incubation at 60° C. for 20 min in a thermomixer (800 rpm, Eppendorf). Completion of the reaction was confirmed by radio-TLC [silica, MeOH:EtOAc=5:95]. The formation of ⁶⁴Cu-ATSM-FITC(6) was confirmed by comparing the HPLC profiles of ⁶⁴Cu-ATSM-FITC(6) and Cu-ATSM-FITC(5) (Waters, Grace Smart RP C18 column [4.6 mm×250 mm, 5 μm], isocratic mobile phase consisting of MeOH:H₂O=60:40, flow rate of 1 mL/min).

### 4. In Vitro Serum Stability

⁶⁴Cu-ATSM-FITC(6) (10 μL, 37 MBq) was first mixed with FBS (500 μL) or PBS (pH 7.4) at the same volume, and the mixture was incubated at 37° C. Demetalization was monitored using radio-TLC (silica, MeOH:EtOAc=5:95) for up to 12 hours.

### 5. Animal Testing

All animal experiments were conducted in accordance with the approved animal protocols and guidelines established by the Kyungpook National University Animal Care Committee (Nos. KNU 2017-0096, 2019-0009, 2019-0101, and 2020-0079).

Male BALB/c mice (6 to 9 weeks old) were purchased from Hyochang Bioscience (Daegu, Korea). All mice were housed at 20 to 24° C. together with sufficient water and commercial food under a 12-h day/night cycle. For studies on H₂S inhibition, male BALB/c mice were injected intraperitoneally with AOAA ((O-(carboxymethyl) hydroxylamine hemihydrochloride, 10 mg/kg, Sigma-Aldrich (C13408-1G)), 1 hour before administered with ⁶⁴Cu-ATSM-FITC(6) into the tail vein.

### 6. Preparation of Neuroinflammation Animal Models

Mice were mounted in a stereotaxic frame under isoflurane anesthesia. Sterile saline (control) or LPS (5 mg/mL, Sigma-Aldrich, Saint Louis, MO, USA, catalog number: L2630) was injected unilaterally into the right striatum (anterior-posterior [AP], 0.5 mm, medial) of each mouse by using a 28-gauge Hamilton syringe attached to an automatic microinjector. Injection was carried out at a rate of 0.2 μL/min for 10 minutes. After each injection, the needle was left in place for 5 minutes before being slowly retracted.

### 7. Study on Biodistribution of ⁶⁴Cu-ATSM-FITC(6) in Normal Mice

BALB/c mice (male, 9 weeks old) were used to investigate the systemic distribution and clearance rate of ⁶⁴Cu-ATSM-FITC (6). ⁶⁴Cu-ATSM-FITC (6) with a radioactivity of 0.56 to 0.74 MBq in a saline solution (5% DMSO, 200 μL) was injected into mice through the tail vein under anesthesia. Mice were sacrificed 5, 30, 1, and 2 hours after the injection (n=4). Blood was drawn, and organs comprising heart, lung, muscle, fat, bone, spleen, kidney, liver, intestine, and brain were harvested, weighed, and analyzed by using a γ-counter (Wallac Wizard 1480, PerkinElmer, France). Radioactivity was calculated as percentage of injected dose per gram (% ID/g).

### 8. Quantification of H₂S in Brain by Methylene Blue Method

The methylene blue method was used to directly measure H₂S concentration in brain tissues. Briefly, PET imaging was performed, brains were harvested, weighed, and homogenized in liquid N₂. An ice-cold PBS solution in a volume equivalent to the weight of the homogenate (w/v, approximately 260 to 300 mg) was added, followed by vortex for 3 min and centrifugation (14,000 rpm, 5 min, 4° C.). Then, 100 μL of the supernatant was incubated together with 100 μL of 1% zinc acetate dihydrate at 37° C. for 10 minutes to immobilize H₂S. 100 μL of 0.1 M sodium tetraborate buffer (pH 9) and 200 μL of 20 mM N, N-dimethyl-p-phenylenediamine sulfate were added to 7.2 M HCl, and 200 μL of 30 mM iron (III) chloride was added to 1.2 M HCl, and the mixture was incubated at 37° C. for 15 min to form methylene blue dyes. The mixture was centrifuged again (14,000 rpm, 5 minutes, 4° C.). 100 μL of supernatant was transferred in triplicate to 96-well plates, and absorbance was recorded spectrophotometrically at 670 nm. The H₂S concentration was calculated by using a standard curve obtained by plotting absorbance (A670) versus NaHS concentration (0 to 30 μM). For quantification of H₂S in the left and right hemispheres of the brains, PET imaging was performed, and only the cerebral hemispheres were harvested and processed separately. The protocols were as described above, except that cold PBS weighed 3 times than each hemisphere (w/v, approximately 50 to 90 mg) was added to the homogenate.

### 9. Animal PET/CT Imaging

Animal PET/CT images were obtained by using a nanoScan PET/CT scanner (PET 82S, Mediso, Budapest, Hungary). Radiolabeled ⁶⁴Cu-ATSM-FITC(6) (˜11 MBq, 200 μL of 5% DMSO/PBS) was injected via tail vein into normal and AOAA-treated BALB/c mice (n=4), and the mice were scanned for 20 minutes at 1 hour after the injection. For PET image-based quantification, regions of interest (ROIs) were manually drawn on the whole brain.

Brain inflammation and control models were prepared 2 days prior to the study on PET imaging (n=5 each). ⁶⁴Cu-ATSM-FITC(6) (˜18 MBq, 200 μL of 5% DMSO/PBS) was injected into mice, and the mice were scanned for 10 minutes at 10 minutes after the injection. In addition, Sprague Dawley rats were injected with radiolabeled ⁶⁴Cu-ATSM-FITC(6) (˜18 MBq, 200 μL of 5% DMSO/PBS) through the tail vein, and the mice were scanned for 20 minutes at 1 hour after the injection. CT images were generated immediately without the use of additional contrast agents. All PET images were reconstructed by using the Mediso Tera-Tomo 3D iterative algorithm, together with corrections for interaction depth, radionuclide decay, detector normalization, decision dead time, and attenuation.

Detection was performed in 1:3 matching smode with four iterations with six subsets. Analysis of the acquired PET/CT images was performed by using the Mediso InterView Fusion software package. The uptake of ⁶⁴Cu-ATSM-FITC(6) in each tissue was normalized to the administered radioactive dose and animal body weight and expressed as the average percent injected dose per gram (% ID/g).

### 10. Histological Analysis of Brain Cytotoxicity Using Cu-ATSM-FITC

Animals were anesthetized by using isoflurane, and injected with 100 μg of Cu-ATSM-FITC(5) in 100 μL of 10% DMSO/PBS, and 500 μg of Cu-ATSM-FITC(5) in 100 μL of 40% DMSO/PBS, respectively, through tail vein injection. The animals were perfused transcardially with saline 1, 3, and 7 days after the injection. The brains were dissected, fixed in 4% paraformaldehyde, and embedded in paraffin to make blocks. Serial coronal sections in paraffin with the thickness of 7 μm were obtained. The sections were stained with H&E by using a staining kit (BBC ClearView, BBC Eosin Y Alcoholic). The stained brain tissues were photographed under a Carl Ziess microscope at 400× magnification.

### 11. Cytotoxicity Analysis of Cu-ATSM-FITC(5) and CuS

The cytotoxicity of Cu-ATSM-FITC(5) was evaluated through 3-(4,5-dimethyl-2-thiazolyl)-2,5-diphenyltetrazolium bromide (MTT; sigma-aldrich) analysis. Human cervical cancer cells (HeLa), human hepatoma cells (HepG2), and human embryonic kidney cells (HEK293) were prepared in 96-well plates at a density of 10,000 cells per well. As compared to the control group treated with PBS alone, they were treated with Cu-ATSM-FITC (5) at increasing concentrations of 1, 10, and 25 μM. Afterwards, they were incubated for 1, 4, and 24 hours (37° C., 5% CO₂), respectively. For each treatment time, each well was added with 10 μL of MTT (5 mg/mL in PBS) solution and incubated for 3 hours. The supernatant was carefully aspirated, and the purple insoluble formazan crystals in the wells were dissolved using DMSO for 30 minutes. Absorbance was measured at 550 nm using a spectrophotometer (SPECTROstar Nano, BMG Labtech, Ortenberg, Germany). CuS was also performed four times in a similar manner by using HeLa, HEK293, HepG2, and human brain glioblastoma (U87MG) cells.

### 12. In Vitro Serum Stability

Radioactively labeled ⁶⁴Cu-ATSM-FITC(6) (10 μL, 37 MBq) was mixed with 100 μL of FBS or PBS at pH 7.4 in the same amount and incubated at 37° C. using a Thermomixer (500 rpm). In addition, demetalization was measured by radio-TLC (silica, MeOH:EtOAC=5:95) every 4 hours for up to 24 hours.

### 13. Measurement of Quantum Yield

The background levels of methanol used in the experiment were measured by using a SpectraMaxi-3 spectraphotometer, and Cu-ATSM-FITC(5) was measured at concentrations of 0.02, 0.04, 0.06, 0.08, and 0.1 μM. A fluorescein standard line graph was obtained according to the concentration and intensity of Cu-ATSM-FITC(5). The quantum yield was calculated according to the following equation: Φ=ΦST (Grad x/Grad ST) (η×2/ηST 2). Here, the subscripts ST and X represent the measured values of the standard and test solutions, respectively. In addition, Φ represents the quantum yield and η represents the refractive index of the solution.

### 14. Preparation of a Rat Model of Parkinson's Disease

The Parkinson's disease-induced rat model was prepared by taking 7-week-old SD rats and injecting 6-hydroxydopamine (6-OHDA) into the medial forebrain bundle using a Hamilton syringe after drilling a small 1.5 mm hole in the rat's skull near the parietal region at 8 weeks of age.

### Experiment Results

The present inventors have designed a novel chelator in which radioactive copper(II)-diacetyl-bis(N4-methylthiosemicarbazone) (ATSM) backbone is directly conjugated to fluorescein isothiocyanate (FITC-NCS). By combining the strengths of two imaging modalities, the high spatial resolution of fluorescence imaging and the unlimited tissue penetration capability of nuclear imaging, we intended to detect H₂S from the cellular level to the whole-body scale using a single imaging probe. Such attempt has never been successful before.

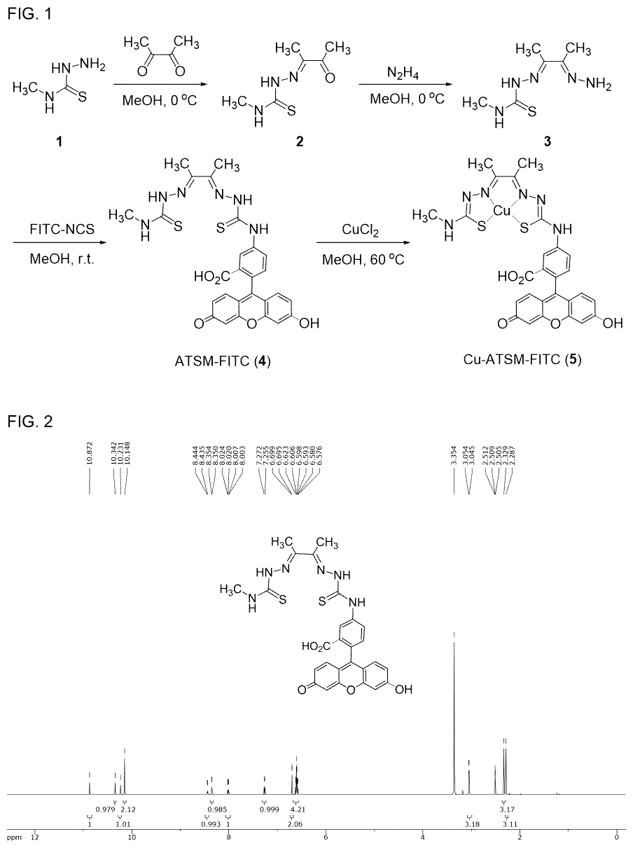

A new chelator, ATSM-FITC (4), was synthesized through four sequential steps (FIG. 1).

Briefly, Intermediate 3 was prepared according to a previously reported procedure and then the terminal amine of Intermediate 3 was conjugated with the NCS group of FITC to obtain the ATSM-FITC conjugate (4). The overall yield of ATSM-FITC conjugate (4) from N-methyl hydrazinecarbothioamide (1) was 25.9%.

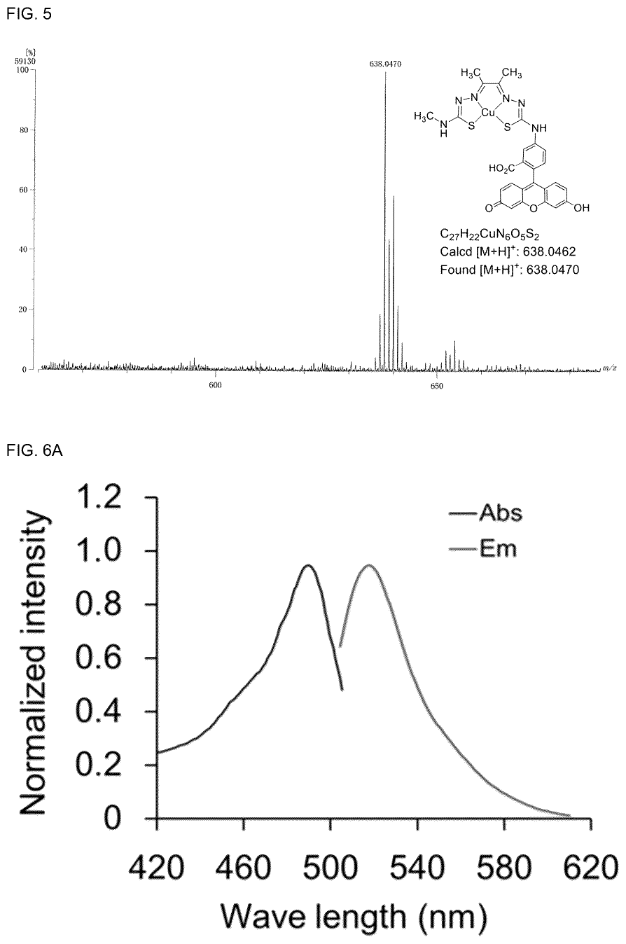

The chemical structure and high purity were confirmed through ¹H and ¹³C NMR spectroscopy and HRMS (FIGS. 2 to 4). ATSM-FITC (4) was refluxed with CuCl₂ in methanol to obtain the Cu(II) complex Cu-ATSM-FITC (5) as a dark brown solid in 33% yield. Purity was determined by HRMS (FIG. 5) and RP-HPLC analysis.

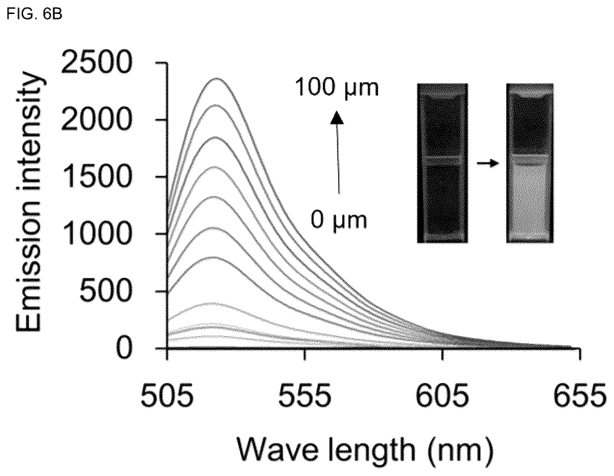

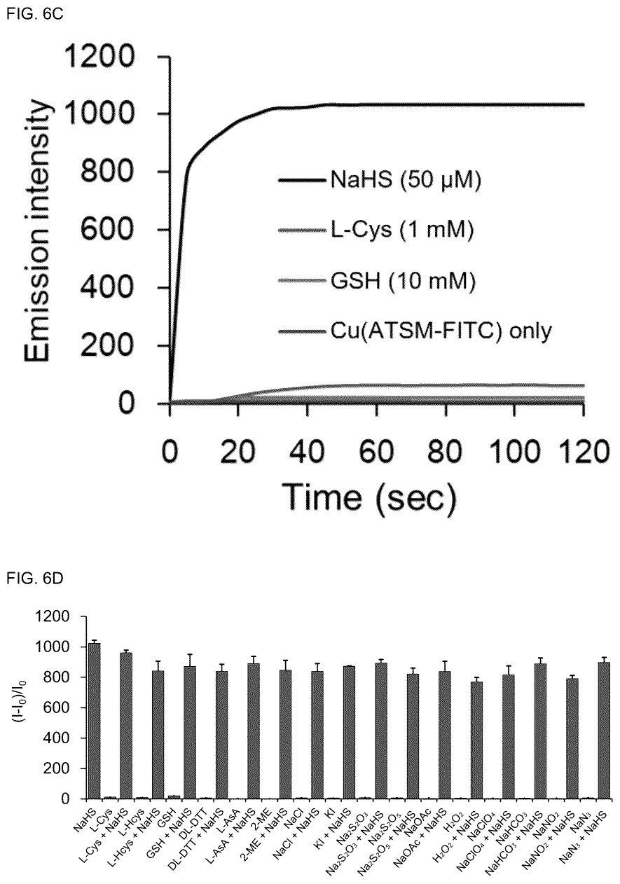

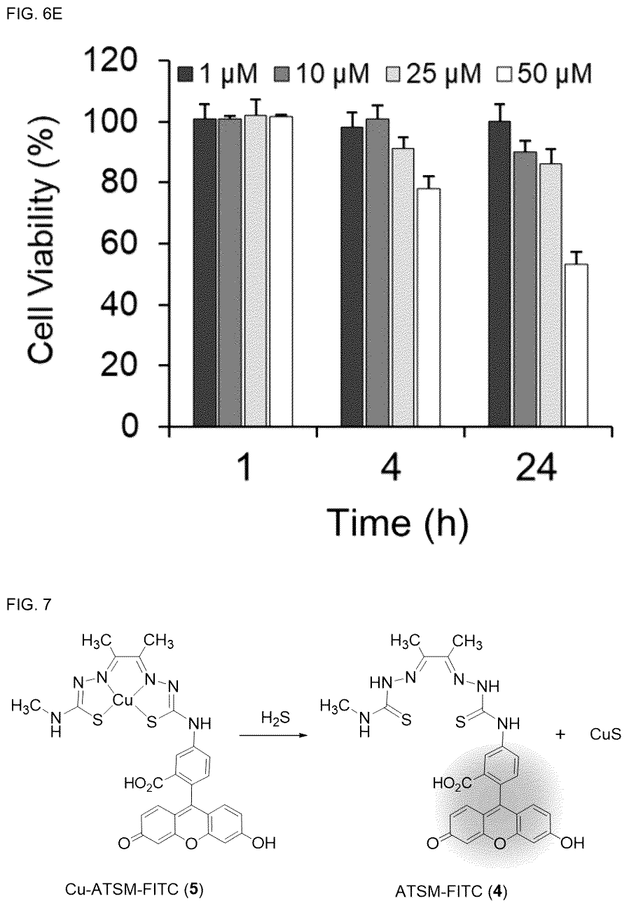

The optical properties of the free ligand ATSM-FITC (4) were evaluated. UV-Vis spectroscopy showed an absorbance band at 494 nm and a maximum fluorescence emission at 524 nm upon excitation at 480 nm (FIG. 6A). The Cu-ATSM-FITC(5) complex did not show a detectable fluorescence signal Since the paramagnetic Cu²⁺ ions coordinated to the ATSM-FITC chelator quenched the fluorescence signal of fluorine-rescein. When H₂S sequestered Cu(II) ions from the copper complex (5) and precipitated CuS, the ATSM-FITC ligand was released and fluorescence was emitted (FIG. 7).

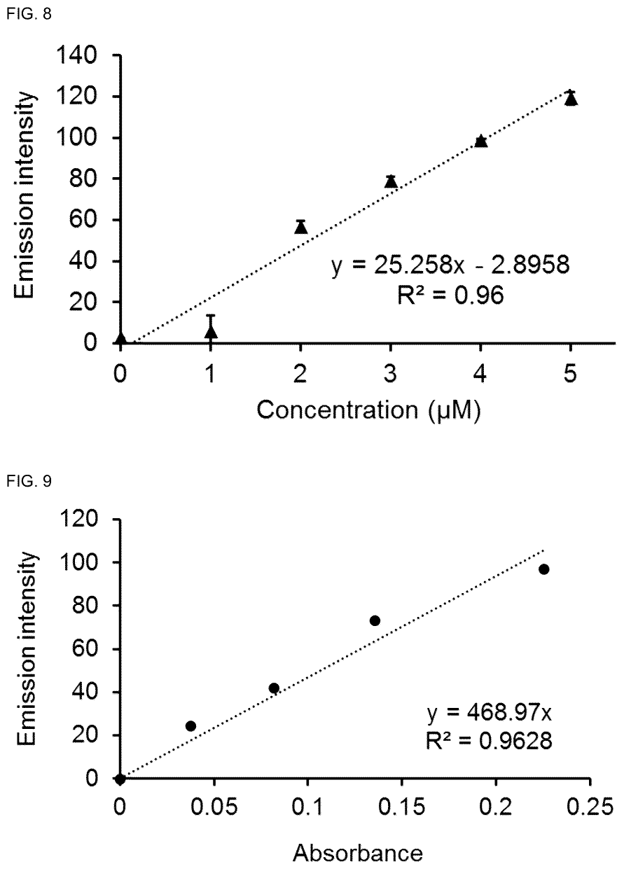

To confirm the sensitivity, Cu-ATSM-FITC(5) was incubated with a wide range of NaHS concentrations (0-100 μM) at 37° C. and the fluorescence intensity was measured (FIG. 6B). The probe showed a gradual increase in fluorescence signals at 524 nm, together with up to 3500-fold fluorescence enhancement at 100 μM of NaHS, as compared to that at the concentration of 0, which is much higher than that reported for most fluorescent H₂S probes. Cu-ATSM-FITC(5) showed rapid reactivity toward H₂S. When reacted with 50 μM NaHS solution, the fluorescence intensity of Cu-ATSM-FITC(5) reached a plateau within 30 seconds (FIG. 6C). In contrast, the reactivity of Cu-ATSM-FITC(5) toward L-cysteine (1 mM) and glutathione (GSH) (10 mM) was minimal, as compared to H₂S, even though their concentrations were at least 20 times higher than H₂S. The detection limit of Cu-ATSM-FITC(5) was calculated to be 0.20 μM using the regression equation (FIG. 8). The quantum yield was confirmed to be 0.33 according to the equation, as compared to the fluorescein standard value (FIG. 9).

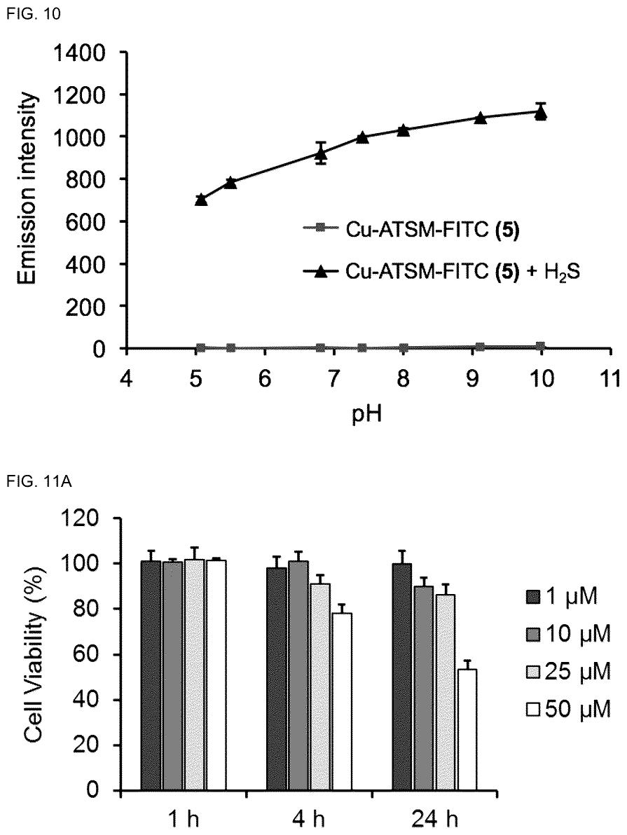

The selectivity of Cu-ATSM-FITC(5) was investigated against a variety of potentially competing biological species in the presence and absence of H₂S (FIG. 6D). Cu-ATSM-FITC(5) did not show reactivity (fluorescence signals) with other species such as biothiols-L-cysteine (Cys), L-homocysteine (Hcys), glutathione (GSH), dithiothreitol (DTT), and 2-mercaptoethanol (2-ME), inorganic sulfur species (S₂O₃²⁻ and S₂O₅²⁻), reducing agents such as ascorbic acid (AA), inorganic and organic nucleophiles (Cl—, I—, OAc—, ClO4-, HCO₃²⁻, N³⁻ or NO²⁻), and reactive oxygen and nitrogen species, including hydrogen peroxide (H₂O₂), peroxynitrite (ONOO⁻), and nitric oxide (NO). However, when NaHS (50 μM) was added to the mixture along with other biologically abundant species, full fluorescence intensity was observed without interference from other biological entities (FIG. 6D). Cu-ATSM-FITC(5) also showed high stability and reactivity over a wide physiological pH range (pH 5 to 10) (FIG. 10). All these results show that Cu-ATSM-FITC (5) has excellent sensitivity, reactivity, and selectivity to be used as a biosensor for H₂S.

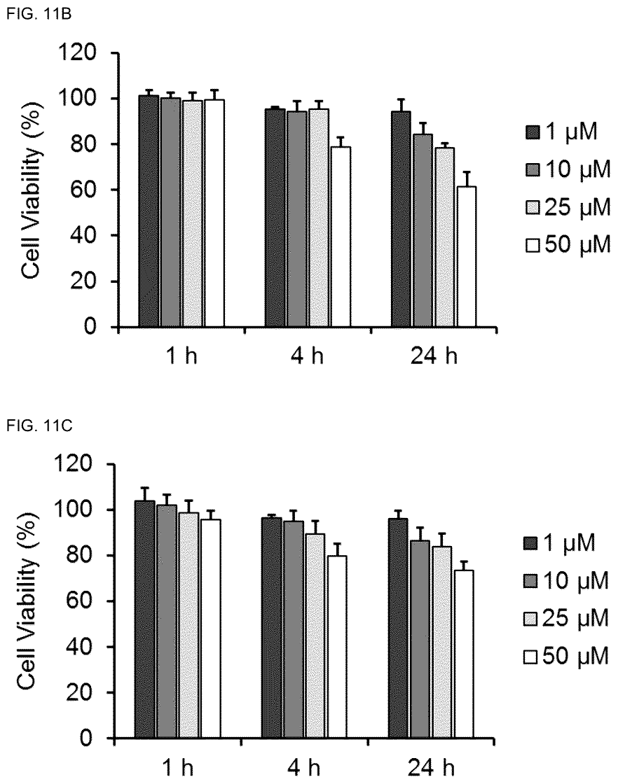

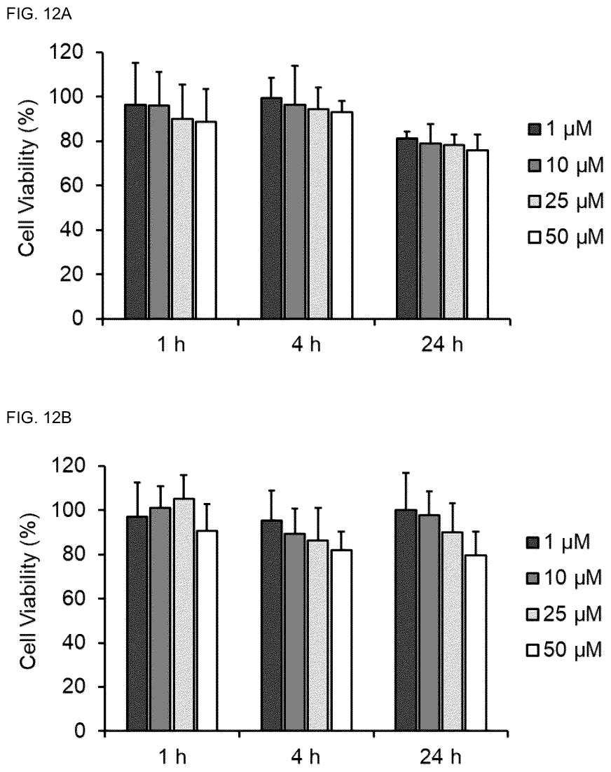

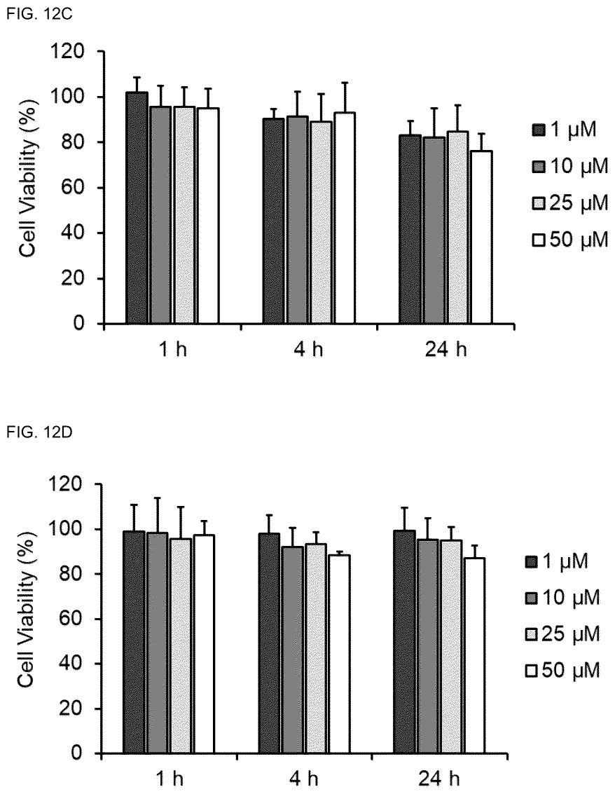

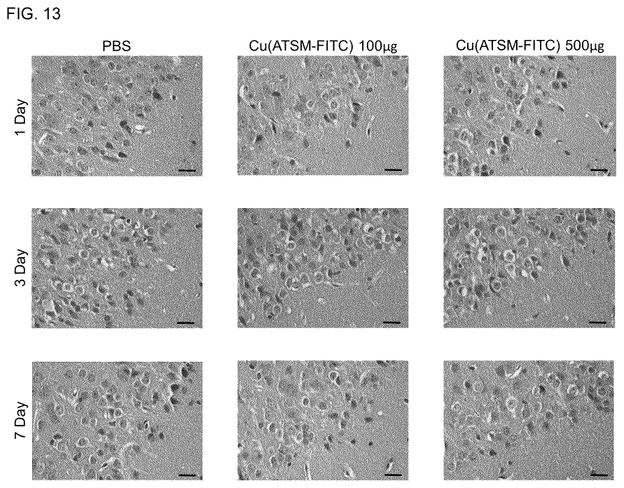

Next, the present inventor tested the toxicity and biocompatibility of Cu-ATSM-FITC(5) before fluorescence imaging of H₂S in HeLa cells. According to MTT analysis, the survival rate of HeLa cells exceeded 85% at concentrations of 1, 10, and 25 μM even after 24 hours of incubation, indicating its excellent biocompatibility (FIG. 6E). No cytotoxicity was observed in other cell lines comprising human hepatoma cell line (HepG2) and human embryonic kidney 293 (HEK293) cells (FIGS. 11A to 11C). In addition, CuS was subjected to MTT analysis in the same manner by adding human brain glioblastoma (U87MG), and, as a result, no cytotoxicity was found in all cell lines (FIGS. 12A to 12D).

H&E staining data of mouse brain tissues confirmed the low toxicity of CuS precipitated for up to 7 days even after injection of a high-dose, 500 μg, of Cu-ATSM-FITC(5) (FIG. 13).

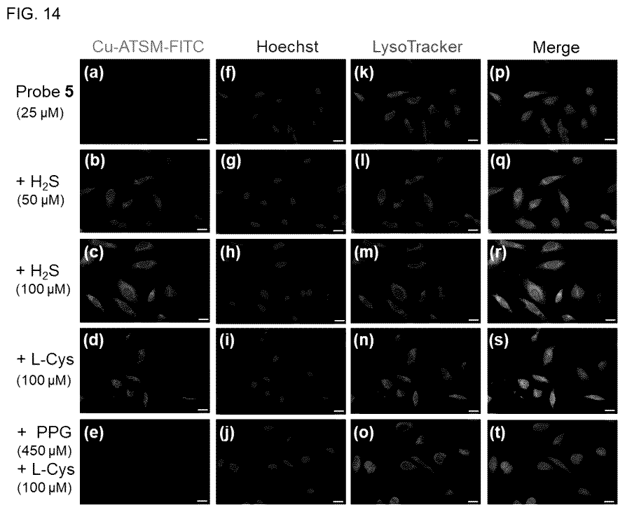

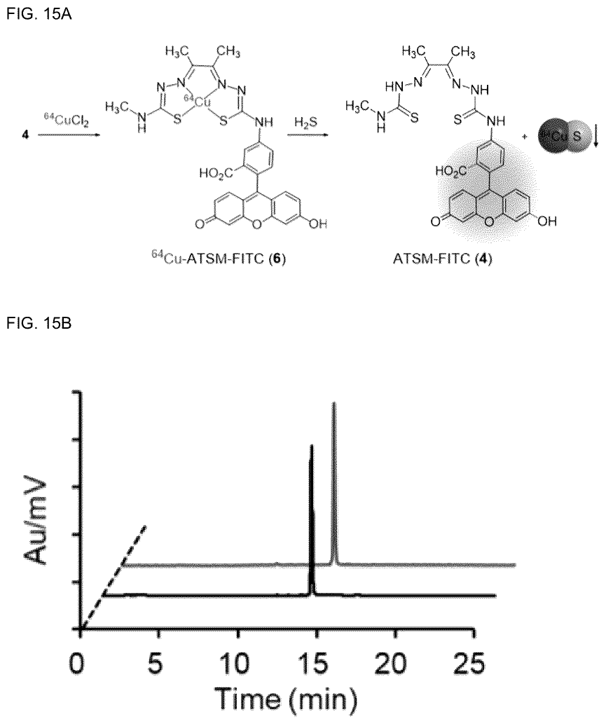

Next, we investigated the usefulness of Cu-ATSM-FITC(5) for monitoring H₂S levels in living cells (FIG. 14). HeLa cells incubated with Cu-ATSM-FITC(5) at a low toxic (25 μM) concentration showed a faint fluorescence signal due to the tightly controlled low levels of H₂S in healthy cells (a). In contrast, a linear rise in green fluorescence intensity was detected upon addition of NaHS at two different concentrations (50 and 100 μM) to probed HeLa cells (b and c). Cu-ATSM-FITC(5) also successfully detected the increase in endogenous H₂S concentration induced by L-cysteine (100 μM) (d). When H₂S-generating cystathionine γ-lyase (CSE) enzyme activity was blocked by DL-propargylglycine (PPG, 450 μM), green fluorescence due to endogenous H₂S was completely reduced (e). In cells co-stained with Hoechst (f-j) and LysoTracker (k-o), an overlap was observed between Cu-ATSM-FITC(5) and LysoTracker (p-t), indicating that Cu-ATSM-FITC(5) predominantly co-stained with lysosomes. Taken together, the fluorescence imaging results indicate that the fluorescent probe Cu-ATSM-FITC (5) can be used to noninvasively monitor small fluctuations in H₂S concentration at the cellular level.

Next, the potential of Cu-ATSM-FITC(5) as a nuclear imaging agent for in vivo imaging was confirmed.

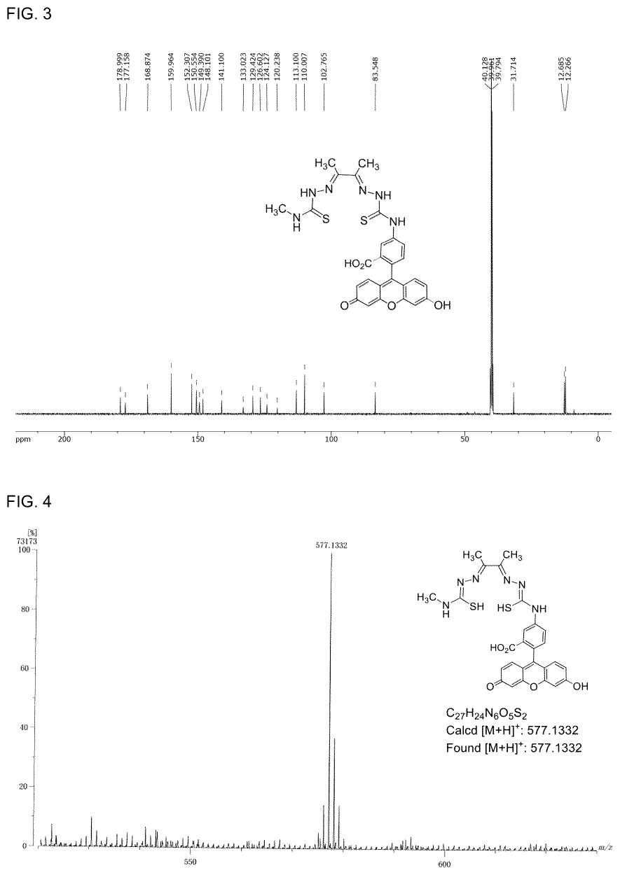

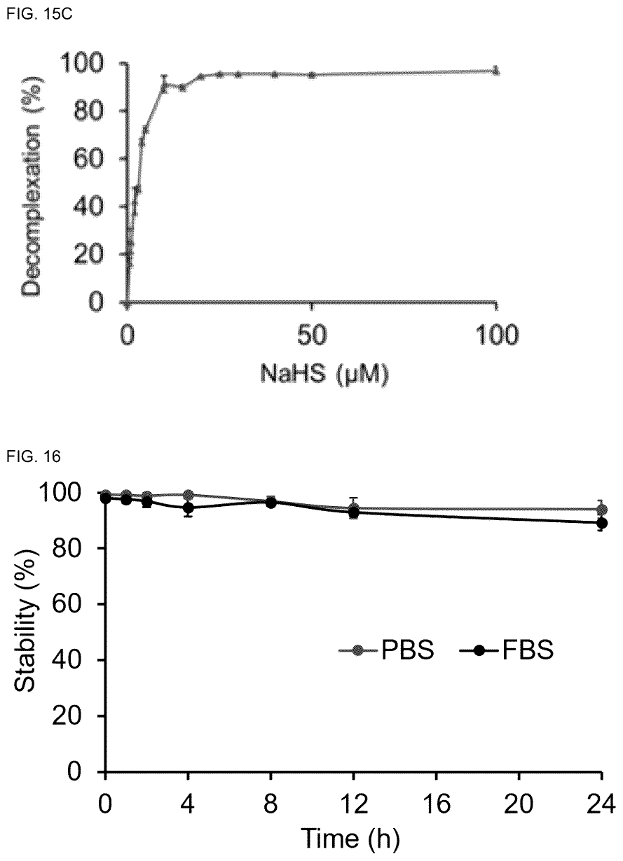

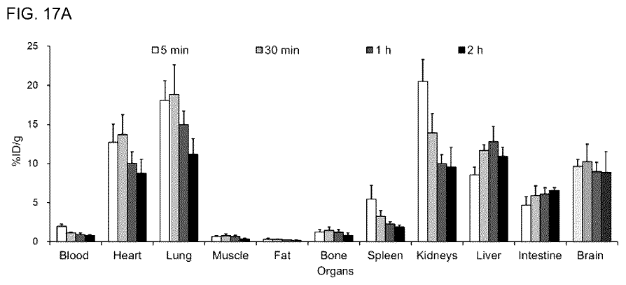

The ligand ATSM-FITC(4) was radiolabeled with ⁶⁴CuCl₂ ions in 0.1M ammonium acetate buffer (pH 6.8) at 60° C., with a radiochemical yield of >97% (FIG. 3). Among the various positron-emitting radioisotopes of copper (⁶⁰Cu, ⁶¹Cu, ⁶²Cu, or ⁶⁴Cu), Cu-64 has a moderately long half-life (12.7 hours) and a decay mode favorable for PET imaging (17.8% β+ decay). The identity of radioactively labeled ⁶⁴Cu-ATSM-FITC(6) was confirmed by comparing the retention time with that of Cu-ATSM-FITC(5), a non-radioactive standard compound, by HPLC (FIG. 15B). ⁶⁴Cu-ATSM-FITC(6) reacted immediately with H₂S, fixing gaseous H₂S into an insoluble copper sulfide (⁶⁴CuS) precipitate (FIG. 15A). The proportion of demetalized ⁶⁴CuS in the intact ⁶⁴Cu-ATSM-FITC(6) complex may be easily quantified by radio-TLC analysis. The Rf value of ⁶⁴Cu-ATSM-FITC(6) was 0.72 on a silica plate with mobile phase (ethyl acetate methanol (95:5)), and ⁶⁴CuS remained at the origin of the TLC plate (FIG. 22). As the concentration of H₂S increased, the decomplexation percentage of ⁶⁴Cu-ATSM-FITC(6) increased proportionally and reached a plateau at 20 μM of H₂S (FIG. 15C). Radiolabeled ⁶⁴Cu-ATSM-FITC (6) showed high stability in both fetal bovine serum (FBS) and phosphate-buffered saline (PBS) solutions at 37° C. Less than 10% degradation of ⁶⁴Cu-ATSM-FITC(6) was observed for up to 24 hours after incubation (FIG. 16). The lipophilicity of the radiotracer was measured using the general octanol-PBS partitioning method.

The log D_{7.4} value of ⁶⁴Cu-ATSM-FITC(6) was 1.70±0.05, and the range of 1.5 to 2.7 is desirable for optimal BBB penetration.

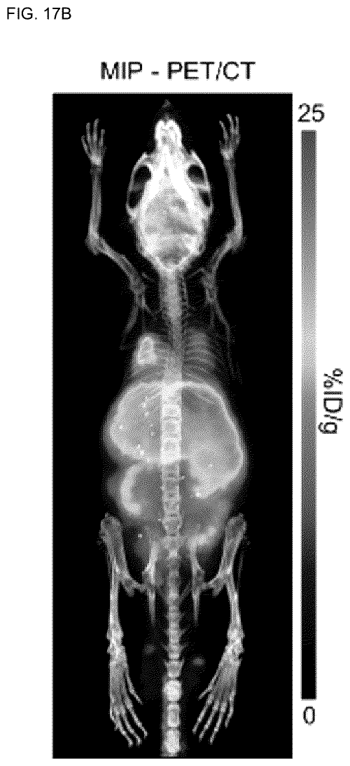

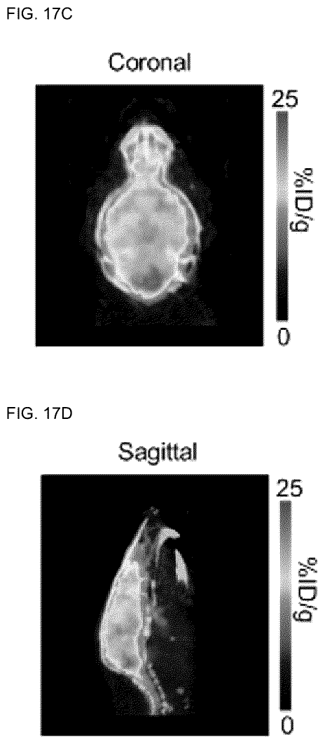

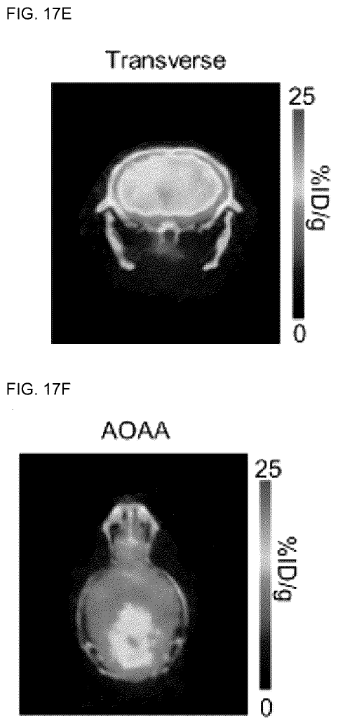

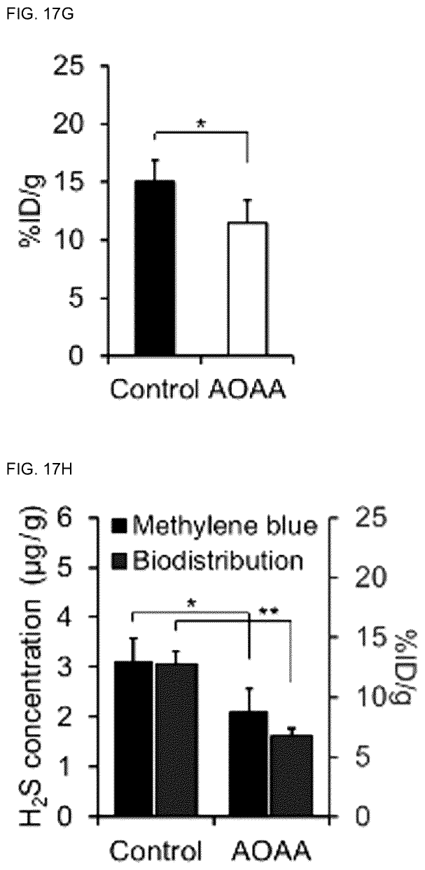

The body distribution and elimination pattern of ⁶⁴Cu-ATSM-FITC(6) was accurately measured in a biodistribution study in normal BALB/c mice (FIG. 17A). High brain uptake was observed as early as 5 minutes after injection. Brain uptake of greater than 9% ID/g at 5 minutes was maintained for up to 2 hours after injection, and more than 3.3% of the total injected activity was found in the whole brain, confirming that it is significantly higher, as compared to existing brain-targeted radio-tracers including ⁶⁴Cu-ATSM (2 to 5% ID/g). The initially high uptake of ⁶⁴Cu-ATSM-FITC(6) in the heart, lungs, and kidneys gradually decreased over time.

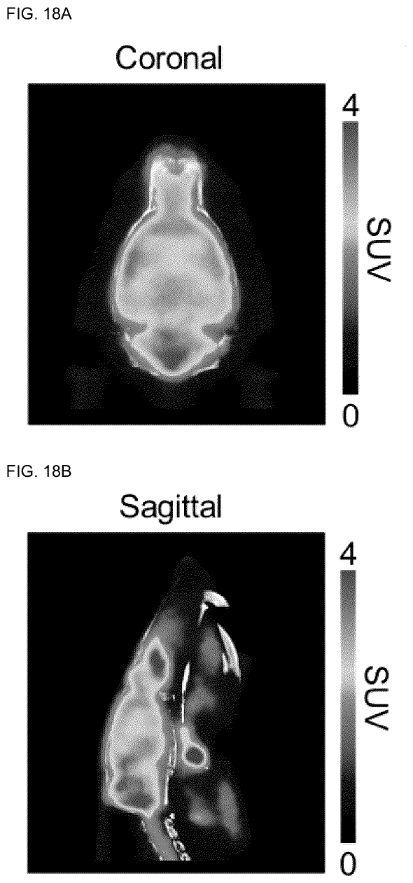

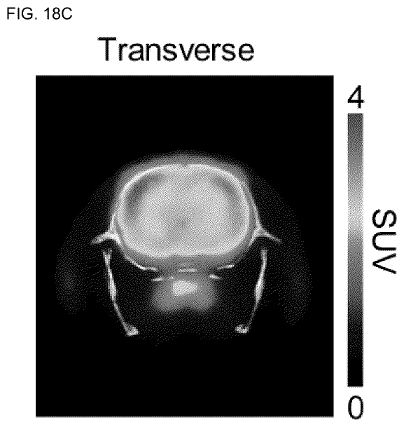

Next, a whole-body imaging study was performed using an animal PET/computed tomography (CT) scanner. The radiotracer ⁶⁴Cu-ATSM-FITC(6) was injected into normal BALB/c mice via the tail vein, and PET/CT images were acquired 1 hour after injection (FIGS. 17B to 17E). As expected from the biodistribution data, significant brain uptake was clearly observed in PET/CT images using Maximum Intensity Projection (MIP) (FIG. 17B). Evenly distributed brain uptake of radioactivity was observed in coronal, sagittal, and cross-sectional images (FIGS. 17C to 17E). In addition, images not only in mice but also in SD-rats were confirmed with the radioactive tracer ⁶⁴Cu-ATSM-FITC(6), and, as a result, similar brain absorption was observed (FIGS. 18A to 18C). However, when the concentration of H₂S in the brain was lowered by intraperitoneal injection of cystathionine β-synthase (CBS), which is an H₂S generating enzyme, and aminooxyacetic acid (AOAA, 10 mg/kg), which is an inhibitor of CSE, the brain uptake of ⁶⁴Cu-ATSM-FITC(6) was significantly reduced (FIG. 17F). In PET imaging, the brain uptake of ⁶⁴Cu-ATSM-FITC(6) was 15.0±1.8 vs. 11.4±2.0% ID/g in control and AOAA-treated mice, respectively (FIG. 17G). The H₂S concentration in the brains of AOAA-treated mice was measured directly by the methylene blue method and was reduced by 32% compared to controls (3.1±0.5 vs. 2.1±0.5 μg/g brain), which is consistent with brain biodistribution data in control and AOAA-treated mice (FIG. 17H).

These data clearly indicate that brain uptake of ⁶⁴Cu-ATSM-FITC(6) is closely correlated with brain H₂S levels.

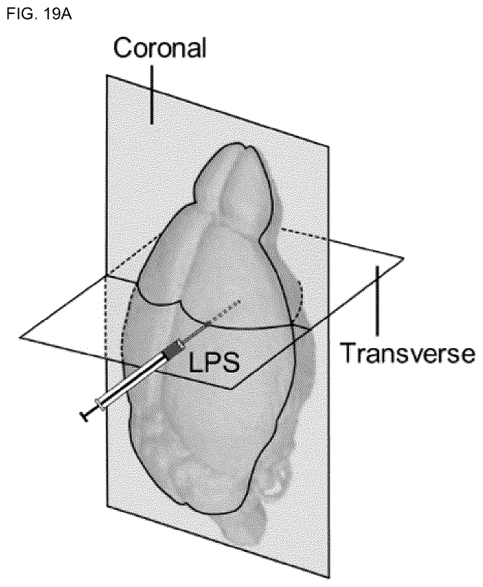

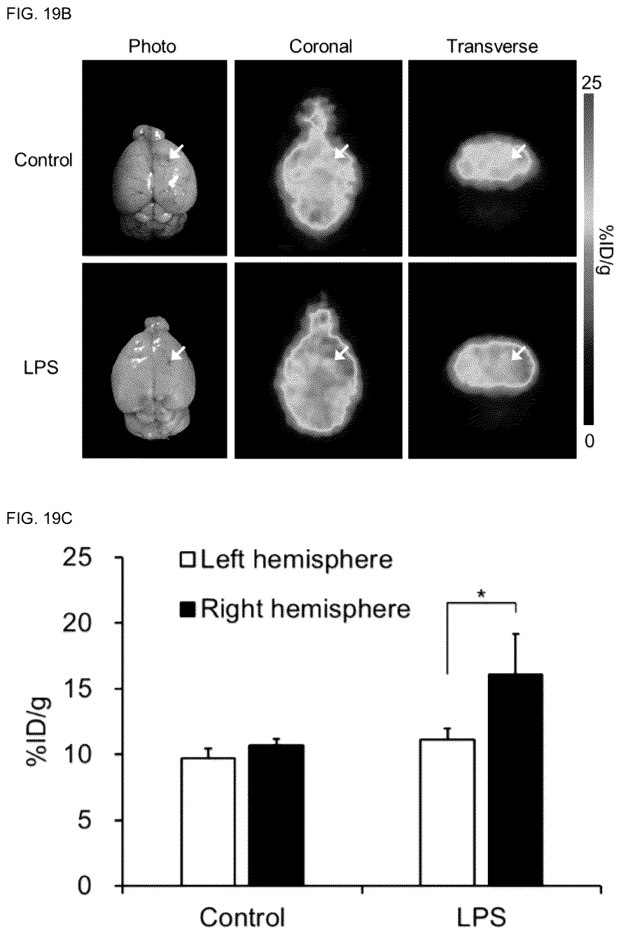

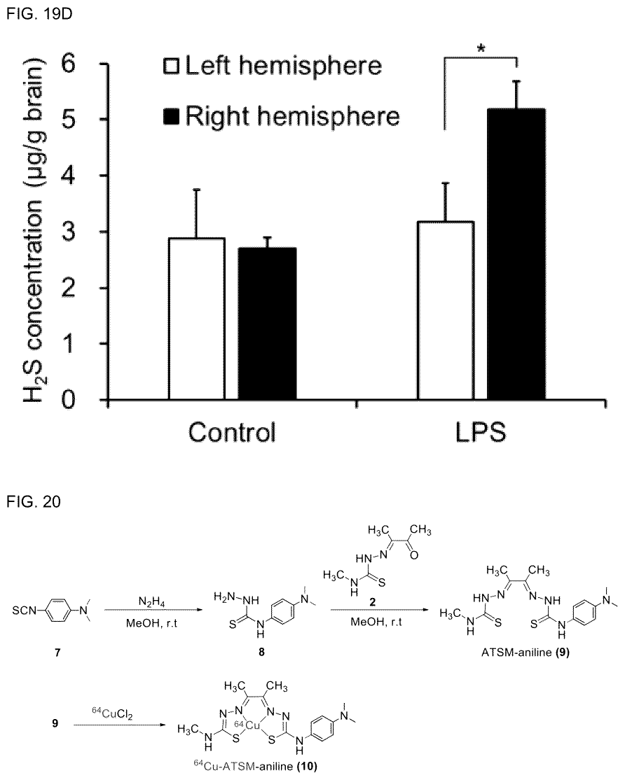

Finally, its potential as an imaging agent for detection of increased H₂S concentrations in disease models was evaluated. Neuroinflammation has been linked to traumatic brain injury, stroke, Alzheimer's disease, Parkinson's disease, and many other brain disorders. In this study, the neuroinflammation model was induced by intracerebroventricular injection of LPS (FIG. 19A).

As a control group, a saline solution was injected instead of LPS. Coronal and transverse PET images showed higher activity in the LPS-treated model, as compared to the control group, especially near the injection site (FIG. 19B). As a result of quantifying the amount of radioactivity in the right hemisphere (injection site) and left hemisphere of the brain by using γ-counter, the absorption amount was similar in both sides in the control group, but a 31% increase was observed at the injection site in LPS-treated animals (FIG. 19C). When H₂S concentrations in the brains were measured ex vivo by the methylene blue method immediately after PET scanning, a 39% increase in H₂S concentrations was detected only in the LPS-treated cerebral hemisphere (4.9±0.5 vs. 3.2±0.7 μg/g brain), which is consistent with PET imaging and biodistribution results.

These PET imaging results clearly show that the radiotracer according to the present invention can detect a sufficiently wide range of H₂S fluctuations (decrease and increase) in the brain.

In addition, we tested whether ⁶⁴Cu-ATSM-FITC(6) can be used to diagnose Parkinson's disease. Several studies have already reported that the concentration and distribution of hydrogen sulfide in the brain changes with the onset and progression of Parkinson's disease. However, due to the lack of a radiolabeled probe that can actually detect hydrogen sulfide in the brain, there have been no reports of hydrogen sulfide-based diagnostic studies for Parkinson's disease. In this study, we used a ⁶⁴Cu-ATSM-FITC radioprobe developed by our group to observe brain uptake changes in a Parkinson's disease model and a normal rat model through PET/CT nuclear medicine imaging studies.

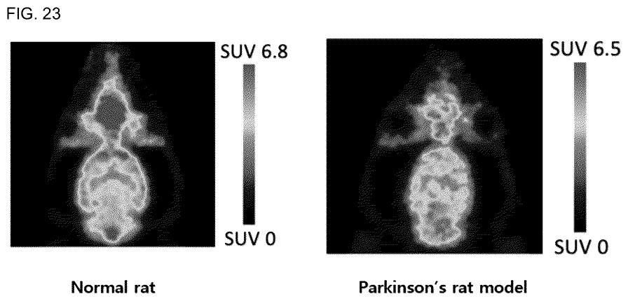

Coronal PET/CT images of ⁶⁴Cu-ATSM-FITC were acquired 1 hour after injection of about 1 mCi of ⁶⁴Cu-ATSM-FITC into the tail vein of normal rats and a Parkinson's disease rat model, and it was found that the uptake in brain regions was significantly lower in the Parkinson's disease rat model. In addition, the uptake pattern was also different between normal rats and the Parkinson's disease rat model (FIG. 23).

### Comparative Data: Body Distribution of ⁶⁴Cu-ATSM-aniline

### (1) Synthesis of (E)-N-(4-(dimethylamino)phenyl)-2-((E)-3-(2-(methylcarbamothioyl)hydrazineylidene)butan-2-ylidene)hydrazine-1-carbothioamide, ATSM-aniline (9)

Compound 8 (420 mg, 2 mmol) in methanol (20 mL) was added dropwise to a cold methanolic solution of Compound 2 (350 mg, 2 mmol, dissolved in 20 mL methanol) in the presence of catalyst HCl. When TLC (silica, MeOH:CH2Cl2=5:95) showed complete consumption of starting material, the mixture was stirred at the same temperature for 1 hour. It was then concentrated under reduced pressure and purified by column chromatography to obtain ATSM-aniline (9) as a yellow solid (260 mg, 36%) (FIG. 20).

### (2) Cu-64 Labeling of ATSM-aniline (9)

A stock solution (1 μg/μL) of the chelator was prepared in anhydrous DMSO. Complexation of ⁶⁴Cu and ATSM-aniline(9) was carried out by reacting in 0.1 M ammonium acetate (pH 6.8, 100 μL) at 60° C. for 20 minutes in a heat mixer (800 rpm). The completion of the reaction was monitored by radio-TLC [silica, MeOH:EtOAc=5:95] (FIG. 20).

### (3) Confirmation of Body Distribution of ⁶⁴Cu-ATSM-aniline

The distribution of ⁶⁴Cu-ATSM-aniline in the body was confirmed by using the same method as described in Experimental Methods 5 and 7 above.

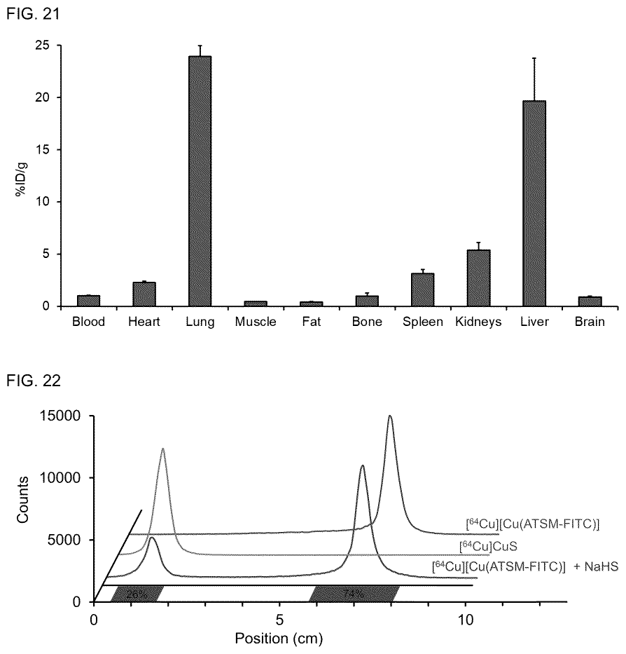

As a result, as shown in FIG. 21, it was confirmed that ⁶⁴Cu-ATSM-aniline was most widely distributed in the lungs and liver, while its distribution in the brain was very low.

For reference, it has been reported that the degree to which Copper Bis(thiosemicarbazonato)-stilbenyl Complexes were absorbed into the brain was very low at 1 to 2% ID/g 2 minutes after injection, and 0.2 to 0.3% ID/g 1 hour after injection (Inorg. Chem. 2020, 59, 16, 11658-11669).

### INDUSTRIAL APPLICABILITY

The complex compound provided by the present invention selectively binds to hydrogen sulfide and can selectively image areas where hydrogen sulfide is abnormally increased within cells or tissues. In particular, it has a very high blood-brain barrier permeability to prevent various neuroinflammatory diseases. It can very effectively detect brain hydrogen sulfide, which is detected at high levels in the brain. In addition, it can be used as a dual-modality contrast agent capable of simultaneous nuclear and fluorescence imaging and, thus, can be very useful for diagnosis and research of various diseases mediated by hydrogen sulfide. Therefore, the industrial applicability of the present invention is very high.

1. A complex compound into which the radioisotope Cu is introduced, defined by the following formula 1: 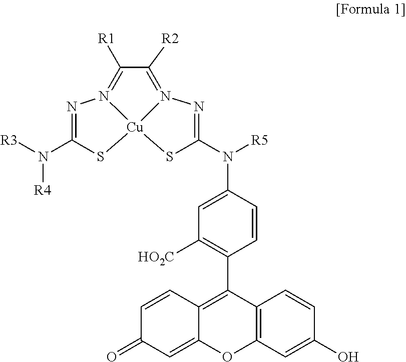
wherein R1 to R5 are each independently hydrogen or C1-C10 straight or branched alkyl, and Cu is ⁶⁰Cu, ⁶¹Cu, ⁶²Cu, ⁶⁴Cu, or ⁶⁷Cu; or a pharmaceutically acceptable salt thereof.

2. The complex compound or pharmaceutically acceptable salt thereof according to claim 1, wherein R1 to R5 are each independently C1-C5 straight alkyl.

3. The complex compound or pharmaceutically acceptable salt thereof according to claim 1, wherein the R1 to R3 are methyl.

4. The complex compound or pharmaceutically acceptable salt thereof according to claim 1, wherein R1 to R3 are methyl, and R4 and R5 are hydrogen.

5. The complex compound or pharmaceutically acceptable salt thereof according to claim 1, wherein the complex compound is a probe for detecting H₂S.

6. The complex compound or pharmaceutically acceptable salt thereof according to claim 1, wherein the lipophilicity log D_{7.4} of the complex compound is 1.5 to 3.0.

7. A contrast agent comprising the complex compound or pharmaceutically acceptable salt thereof according to claim 1.

8. The contrast agent according to claim 7, wherein the contrast agent is one or more selected from the group consisting of a contrast agent for positron emission tomography (PET), a contrast agent for fluorescence imaging, a contrast agent for gamma cameras, a contrast agent for single photon emission computed tomography (SPECT), a Cherenkov optical imaging contrast agent, and a contrast agent for charge-coupled device (CCD).

9. The contrast agent according to claim 7, wherein the contrast agent is a dual-modality contrast agent capable of simultaneous PET and fluorescence imaging.

10. The contrast agent according to claim 7, wherein the contrast agent is used to detect hydrogen sulfide H₂S in the body of a subject.

11. The contrast agent according to claim 7, wherein the contrast agent is used to detect hydrogen sulfide H₂S in the brain of a subject.

12. A composition for diagnosing inflammatory diseases, comprising the complex compound or pharmaceutically acceptable salt thereof according to claim 1.

13. The composition for diagnosing inflammatory diseases according to claim 12, wherein the inflammatory disease is one or more types selected from the group consisting of neuroinflammatory diseases, rheumatoid arthritis, non-rheumatic inflammatory arthritis, arthritis related to Lyme disease, inflammatory osteoarthritis, meningitis, osteomyelitis, inflammatory bowel disease, appendicitis, pancreatitis, sepsis, pyelitis, nephritis, and inflammation diseases due to bacterial infection.

14. The composition for diagnosing inflammatory diseases according to claim 13, wherein the neuroinflammatory disease is selected from the group consisting of Alzheimer's disease, vascular dementia, frontotemporal dementia, alcoholic dementia, Parkinson's disease, traumatic brain injury, Niemann-Pick disease, amyotrophic axial sclerosis, multiple sclerosis, Huntington's disease, Creutzfeldt-Jakob disease and stroke.

15. Use of the complex compound or pharmaceutically acceptable salt thereof according to claim 1 in preparing a composition for diagnosing inflammatory diseases.

16. A method for diagnosing inflammatory diseases, comprising:
(a) administering the complex compound or pharmaceutically acceptable salt thereof according to claim 1 to a subject;
(b) scanning the subject by using a fluorescence imaging device, a radioactive imaging device, or a combination thereof; and
(c) analyzing the scanned image and diagnosing the subject as having an inflammatory disease when a fluorescence signal, a radiation signal, or a combination thereof is increased, as compared to the control group.
